(19) United States
(12) Patent Application Publication
(10) Pub. No.: US 2023/0368925 A1
(43) Pub. Date: Nov. 16, 2023

(54) MAPPING BIOMEDICAL ENTITIES

(71) Applicant: Innoplexus AG, Eschborn (DE)

(72) Inventors: Gaurav Tripathi, Pune (IN); Om Sharma, Dhanbad (IN); Vatsal Agarwal, Rampur (IN); Anurag Chandrekar, Raipur (IN)

(21) Appl. No.: 18/219,683

(22) Filed: Jul. 9, 2023

Related U.S. Application Data
(63) Continuation-in-part of application No. 16/366,451, filed on Mar. 27, 2019.

(30) Foreign Application Priority Data
Mar. 27, 2018 (GB) 1804894.2

(51) Int. Cl. G16H 50/70; G16H 70/40; G16H 70/60; G16H 15/00

(52) U.S. Cl. CPC G16H 50/70; G16H 70/40; G16H 70/60; G16H 15/00; G16H 20/10

(57) ABSTRACT

An approach is disclosed for mapping biomedical entities, wherein each of the biomedical entities belong to one of a predefined class: target, disease, pathway, and drug. The system comprises a database arrangement operable to store existing data sources; a processing module communicably coupled to the database arrangement. The processing module operable to receive a user-input of a biomedical entity belonging to one of the predefined classes, wherein the predefined class of the biomedical entity defines an input class. Extract a plurality of biomedical entities related to the biomedical entity of the user-input from existing data sources, wherein the plurality of biomedical entities belong to predefined classes except the input class. Identify at least one pair of biomedical entities, from the plurality of extracted biomedical entities, having an association there between and map the plurality of biomedical entities to the biomedical entity of the user-input.

If an Application Data Sheet (ADS) has been filed for this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)). In addition, the present application is related to the “Related Applications,” if any, listed below.

### BACKGROUND

The present disclosure relates generally to data processing; and more specifically, to bioinformatics.

### SUMMARY

According to an embodiment of the present invention, there is a method for dynamically identifying associations between a first biomedical entity and a second biomedical entity. The first biomedical entity is received via a user interface. The first biomedical entity is mapped to a first class in a set of predefined classes wherein each entry in the set of predefined classes is one of a target, a disease, a pathway, and a drug. A plurality of biomedical entities related to the first biomedical entity is extracted from existing data sources, wherein the plurality of biomedical entities belong to one of the predefined classes except the first class. The extracted plurality of biomedical entities is stored in a repository. At least one pair of biomedical entities is identified from the plurality of extracted biomedical entities, and wherein the at least one pair has a first entry and a second entry with the first entry having an association with the second entry, and wherein the second entry belongs to one of the predefined classes except the first class. The user interface presents a representation of the at least one pair of biomedical entities.

In one aspect, an embodiment of the present disclosure provides a system that maps biomedical entities, wherein each of the biomedical entities belongs to one of a predefined class: target, disease, pathway, and drug, wherein the system includes a computer system, characterized in that the system comprises:
a database arrangement operable to store existing data sources; and
a processing module communicably coupled to the database arrangement, the processing module operable to:
receive a user-input of a biomedical entity belonging to one of the predefined classes, wherein the predefined class of the biomedical entity defines an input class;
extract a plurality of biomedical entities related to the biomedical entity of the user-input from existing data sources, wherein the plurality of biomedical entities belongs to predefined classes except the input class;
identify at least one pair of biomedical entities, from the plurality of extracted biomedical entities, having an association there between, wherein each biomedical entity of the at least one pair of biomedical entities belongs to different predefined classes; and
map the plurality of biomedical entities to the biomedical entity of the user-input.

In another aspect, an embodiment of the present disclosure provides a method of mapping biomedical entities, wherein each of the biomedical entities belongs to one of a predefined class: target, disease, pathway, and drug, wherein the method includes using a computer system, characterized in that the method comprises:
providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belong to predefined classes except the input class;
identifying at least one pair of biomedical entities, from the plurality of extracted biomedical entities, having an association there between, wherein each biomedical entity of the at least one pair of biomedical entities belongs to different predefined classes; and
mapping the plurality of biomedical entities to the biomedical entity of the user-input.

In yet another aspect, an embodiment of the present disclosure provides a computer readable medium containing program instructions for execution on a computer system, which when executed by a computer, cause the computer to perform method steps for mapping biomedical entities, wherein each of the biomedical entities belongs to one of a predefined class: target, disease, pathway, and drug, the method comprising the steps of:
providing a user-input of a biomedical entity belonging to one of the predefined classes, wherein the predefined class of the biomedical entity defines an input class; —extracting a plurality of biomedical entities related to the biomedical entity of the user-input from existing data sources, wherein the plurality of biomedical entities belong to predefined classes except the input class;
identifying at least one pair of biomedical entities, from the plurality of extracted biomedical entities, having an association there between, wherein each biomedical entity of the at least one pair of biomedical entities belongs to different predefined classes; and
mapping the plurality of biomedical entities to the biomedical entity of the user-input.

### BRIEF DESCRIPTION OF THE DRAWINGS

The present invention may be better understood, and its numerous objects, features, and advantages made apparent to those skilled in the art by referencing the accompanying drawings, wherein:

FIG. 1 illustrates steps of a method of mapping biomedical entities, in accordance with an embodiment of the present disclosure;

FIG. 2 is a block diagram of a system that maps biomedical entities, in accordance with an embodiment of the present disclosure;

FIG. 3-4 are schematic illustrations of exemplary implementations of the method of mapping biomedical entities, in accordance with an embodiment of the present disclosure; and

FIG. 5 depicts on overview of an analysis of advantages versus disadvantages of a relationship identified between a pair of mapped biomedical entities; and

FIG. 6 depicts a user interface process in accordance with an embodiment of the present disclosure.

### DETAILED DESCRIPTION

The present disclosure also relates to systems that maps associations between biomedical entities. Moreover, the present disclosure relates to methods for mapping associations between biomedical entities. Moreover, the present disclosure also relates to computer readable medium containing program instructions for execution on a computer system, which when executed by a computer, cause the computer to perform method steps for mapping biomedical entities.

In recent years, increased population and pollution have led to an unprecedented growth of diseases across the globe. In order to overcome the array of challenges, drug discoveries are advancing rapidly, research and experiments are also going on a regular basis. With the advent of technology and breakthrough research in field of medicines, a number of medications and treatment are available for treating health ailments. Generally, researchers and physicians study several biomedical entities relating to human anatomy and pharmaceutical compounds. Furthermore, associations between such biomedical entities provides invaluable insights relating to diseases and treatments.

Furthermore, due to a rapid increase in the number of research publications, clinical trials, research related to such biomedical entities using relevant existing information from has become challenging.

Furthermore, currently available techniques access information regarding such biomedical entities from Internet or several other existing data sources. However, such data sources may lack in information and may not comprise relevant updated information.

Furthermore, a user may need to access several data sources in order to retrieve relevant information related to the biomedical entities. Therefore, the currently available techniques for accessing data may require extensive input from the user. Additionally, the conventional techniques provide ambiguous information related to the biomedical entities and associations there between. Consequently, the currently available techniques for accessing information regarding medication, disease, and other factors associated thereto do not provide optimal, adequate and centralized information. Therefore, in light of the foregoing discussion, there exists a need to overcome the aforementioned drawbacks associated with accessing data related to biomedical entities.

The present disclosure seeks to provide a system that maps the biomedical entities, wherein each of the biomedical entities belongs to one of a predefined class: target, disease, pathway, drug. The present disclosure also seeks to provide a method of mapping biomedical entities. The present disclosure also seeks to provide a computer readable medium containing program instructions for execution on a computer system, which when executed by a computer, cause the computer to perform method steps for mapping biomedical entities. Furthermore, the present disclosure seeks to provide a solution to the existing problem of redundant, unorganized and unmanageable biomedical data. Moreover, the present disclosure provides an optimal way of substantially reducing effort required in accessing biomedical data. An aim of the present disclosure is to provide a solution that overcomes at least partially the problems encountered in prior art and provides an efficient method and system for mapping biological entities.

Embodiments of the present disclosure substantially eliminate or at least partially address the aforementioned problems in the prior art, and enables an efficient, effective, seamless, structured and optimal method of mapping associations among biomedical entities including: target, disease, pathway, drug.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers. Embodiments of the present disclosure are now be described, by way of example only, with reference to the following diagrams.

In overview, embodiments of the present disclosure are concerned with mapping biomedical entities and specifically to, determining associations of a given biomedical entity in a pharmaceutical network. The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a system that maps biomedical entities, wherein each of the biomedical entities belongs to one of a predefined class: target, disease, pathway, and drug, wherein the system includes a computer system, characterized in that the system comprises:
a database arrangement operable to store existing data sources; and
a processing module communicably coupled to the database arrangement, the processing module operable to:
receive a user-input of a biomedical entity belonging to one of the predefined classes, wherein the predefined class of the biomedical entity defines an input class;
extract a plurality of biomedical entities related to the biomedical entity of the user-input from existing data sources, wherein the plurality of biomedical entities belong to predefined classes except the input class;
identify at least one pair of biomedical entities, from the plurality of extracted biomedical entities, having an association there between, wherein each biomedical entity of the at least one pair of biomedical entities belongs to different predefined classes; and
map the plurality of biomedical entities to the biomedical entity of the user-input.

In another aspect, an embodiment of the present disclosure provides a method of mapping biomedical entities, wherein each of the biomedical entities belong to one of a predefined class: target, disease, pathway, and drug, wherein the method includes using a computer system, characterized in that the method comprises: providing a user-input of a biomedical entity belonging to one of the predefined classes, wherein the predefined class of the biomedical entity defines an input class;
extracting a plurality of biomedical entities related to the biomedical entity of the user-input from existing data sources, wherein the plurality of biomedical entities belong to predefined classes except the input class;
identifying at least one pair of biomedical entities, from the plurality of extracted biomedical entities, having an association there between, wherein each biomedical entity of the at least one pair of biomedical entities belongs to different predefined classes; and
mapping the plurality of biomedical entities to the biomedical entity of the user-input.

The present disclosure provides the aforementioned system and method for mapping biomedical entities. The described method maps associations among biomedical entities including: target, disease, pathway, and drug. Thus, the method provides relevant and accurate associations between a pair of biomedical entities. The described method does not require users to exert manual effort in accessing biomedical entities associated with the biomedical entity of user-input.

Consequently, the present disclosure provides an effortless and less time consuming solution for retrieving biomedical data. Furthermore, the method provides a common platform for accessing relevant biomedical data associated to biomedical entity of user-input.

The computer system relates to at least one computing unit comprising a central storage system, processing units and various peripheral devices. Optionally, the computer system relates to an arrangement of interconnected computing units, wherein each computing unit in the computer system operates independently and may communicate with other external devices and other computing units in the computer system.

The term “system that maps” is used interchangeably with the term “system for mapping”, wherever appropriate i.e. whenever one such term is used it also encompasses the other term.

Throughout the present disclosure, the term “biomedical entities” refers to a therapeutic data unit related to biomedical sciences. Furthermore, the biomedical entities have an association there between based on functional aspect thereof. For example, the biomedical entity “Nexium” may be used to reduce production of stomach acid in human body, wherein “stomach acid” may be another biomedical entity. Furthermore, the biomedical entities and associations thereof are analysed to determine diagnosis, monitoring and therapy of a specific disease associated thereto. Additionally, the biomedical entities are mapped with related one or more biomedical entities in order to identify associations there between.

Furthermore, the term “mapping” used herein refers to determination of direct or indirect association (namely, relationship) between two or more biomedical entities. Furthermore, mapping is performed among biomedical entities having different features. Beneficially, mapping of biomedical entities provide non-ambiguous and non-redundant determination and representation of associations between biomedical entities. Furthermore, each of the biomedical entities belongs to one of a predefined class: target, disease, pathway and drug.

Throughout the present disclosure, the term “predefined class” refers to a specific group of entities having similar characteristics. Furthermore, the biomedical entities in one of the predefined class may have an association with one or more biomedical entities associated with any of the other predefined classes. For example, an anti-allergy medicine “cetirizine” may belong to predefined class: drug. Furthermore, the anti-allergy medicine “cetirizine” may be associated with a biomedical entity “allergy” belonging to predefined class: disease. Moreover, biomedical entities belonging to the predefined class: target, are mainly enzymes and/or proteins. Specifically, the biomedical entities belonging to the predefined class: target, are biological sites generally associated with target sites for pharmaceutical drugs. More specifically, malfunctioning or anomaly in working of such biological site may cause a disease in a body (for example, a human body). In an example, “epidermal growth factor receptor” may be a protein belonging to the predefined class: target. Furthermore, a biomedical entity belonging to the predefined class: target has an association with one or more biomedical entities belonging to the predefined classes: disease, pathway and drug.

Furthermore, the predefined class: disease, includes biomedical entities with properties that cause adverse physiological effects on a subject (such as a human, animal and the like). The biomedical entities belonging to the predefined class: disease, have associations with one or more biomedical entities of the predefined classes: target, drug and pathway. In an example, a biomedical entity “Jaundice” belonging to the predefine class: disease, may have associations with biomedical entities “Bilirubin” belonging to the predefined class: target, and “phenobarbital” belonging to the predefined class: drug. Additionally, a biomedical entity belonging to the predefined class: disease, has associations with one or more biomedical entity belonging to the predefined class: pathway. Moreover, the predefined class: pathway, relates to a collection (namely, series, route) of molecular regulators, chemical reactions, series of molecular events and so forth that lead to a certain product or physiological changes in the subject. Subsequently, such changes affect one or more biomedical entities, belonging to the predefined class: target, causing another biomedical entity belonging to the predefined class: disease.

Additionally, the predefined class: drug, includes biomedical entities such as a medicine, chemical compound, substance and the like that has a physiological effect when ingested, injected or otherwise introduced into the body. Furthermore, biomedical entities that belong to the predefined class: drug, have properties affecting a biomedical entity belonging to the predefined class: target. Additionally, such an affect reaches the biomedical entity belonging to the predefined class: target, through a biomedical entity belonging to the predefined class: pathway. Subsequently, such affect results in curing yet another specific biomedical entity belonging to the predefined class: disease.

As mentioned previously, the method of mapping biomedical entities comprises providing the user-input of the biomedical entity belonging to one of the predefined classes, wherein the predefined class of the biomedical entity defines the input class. Specifically, the processing module is operable to receive the user-input of the biomedical entity belonging to one of the predefined classes, wherein the predefined class of the biomedical entity defines the input class. Furthermore, the user-input is a biomedical entity belonging to any one of the predefined class: target, disease, pathway and drug. Additionally, the predefined class associated with the user-input biomedical entity is the input class. Specifically, at an instance the input class may be any one of the predefined class: target, disease, pathway and drug. In an example, the user input may be a biomedical entity “breast neoplasms”. Moreover in the example, the biomedical entity of the user input belongs to the predefined class: disease. Therefore in such example, the input class is defined as the predefined class: disease. Moreover, the processing module is configured to receive the user-input using a user interface, drop down menu, command prompt and so forth. Furthermore, the term “processing module” as used herein, relates to a computational element that is operable to respond to and process instructions. Optionally, the processing module includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processing module” may refer to one or more individual processors, processing devices and various elements associated with a processing device that may be shared by other processing devices. Additionally, the one or more individual processors, processing devices and elements are arranged in various architectures for responding to and processing the instructions.

Therefore, the processing module is communicably coupled to the database arrangement. Furthermore, the database arrangement is operable to store the existing data sources. Additionally, the term “existing data sources” as used herein, relates to organized or unorganized sources of digital information regardless of the manner in which the information is represented therein. Specifically, such digital information is related to biomedical entities namely, target, disease, pathway and drug. Furthermore, the existing data sources may be publicly available internet sources. For example, the existing data sources may include research publications, clinical trials, company websites, blogs, news websites, research institute websites, government websites, online surveys and so forth. Additionally, the existing data sources include biomedical entities and associated information thereof related to previous user-input of biomedical entity.

The processing module is communicably coupled to the database arrangement. Furthermore, the processing module may be coupled to the database arrangement using a network. Moreover, the network may relate to an arrangement of interconnected programmable and/or non-programmable components that are configured to facilitate data communication between one or more electronic devices, software modules and/or databases, whether available or known at the time of filing or as later developed. Additionally, the network employs wired or wireless communication that can be carried out via any number of known protocols.

As mentioned previously, the method of mapping biomedical entities further comprises extracting the plurality of biomedical entities related to the biomedical entity of the user-input from existing data sources, wherein the plurality of biomedical entities belong to predefined classes except the input class. Specifically, the processing module is operable to extract the plurality of biomedical entities related to the biomedical entity of the user-input from existing data sources, wherein the plurality of biomedical entities belong to predefined classes except the input class. Furthermore, biomedical entities associated to the user-input of the biomedical entity belong to one of the predefined class. Subsequently, the plurality of biomedical entities related to the biomedical entity of the user-input belongs to any of the predefined classes except the predefined class identical to the input class. In an instance when the input class is drug, the extracted plurality of biomedical entities may belong to the predefined class: target, disease and pathway. In an example, when user-input is a biomedical entity “EGFR” belonging to the predefined class: target. Consequently, the predefined class: target is defined as the input class. Additionally, plurality of biomedical entities may be extracted, wherein the plurality of biomedical entities belong to the predefined classes except for the input class namely, disease, pathway and drug. The extracted plurality of biomedical entities may include “MAPK Signalling” belonging to predefined class: pathway, “Lung Neoplasm” belonging to predefined class: disease and “Trastuzumab” belonging to the predefined class: drug.

Optionally, when input class is predefined class: drug, each of the extracted plurality of biomedical entities may belong to one of the predefined classes: target, disease and pathway. More optionally, when input class is predefined class: disease, each of the extracted plurality of biomedical entities may belong to one of the predefined classes: target, drug and pathway. Additionally, when input class is predefined class: pathway, each of the extracted plurality of biomedical entities may belong to one of the predefined classes: target, disease and drug.

Optionally, the method further comprises tagging the biomedical entities in the predefined class: drug, with one of the tags: approved, investigational, combinational, and potential. Furthermore, the processing module is operable to tag the biomedical entities in the predefined class: drug, with one of the tags: approved, investigational, combinational, and potential. Specifically, tagging the biomedical entities in the predefined class drug provides additional information associated with a specific biomedical entity. Beneficially, such tagging may provide verified information associated with specific drugs. Moreover, tagging the biomedical entities in the predefined class drug with the tag “approved” indicates that the drug may be well known and frequently used in treatment of disease associated thereto. In an example, “Trastuzumab”, that is a known drug for treating “breast neoplasm”, may be tagged with the tag approved. Additionally, tagging the biomedical entities in the predefined class drug with the tag “investigational” indicates that the tagged biomedical entities may be under clinical trials. Consequently, effects of such biomedical entities may be uncertain. In another example, “BNC-105”, that is a drug in clinical trial for treatment of “breast neoplasm”, may be tagged with the tag “investigational”. Furthermore, tagging the biomedical entities in the predefined class drug with the tag “combinational” indicates that the tagged biomedical entities may have two or more active pharmaceutical ingredients combined in a single dosage form, which may be manufactured and distributed in fixed doses. In yet another example, “Ceftriaxone+Levofloxacin” may be two active pharmaceutical ingredients used in appropriate ration for treating “breast neoplasm”, may be tagged with the tag “combinational”. Furthermore, tagging the biomedical entities in the predefined class drug with the tag “potential” indicates that the tagged biomedical entities are approved drugs for one or more other biomedical entities belonging to the predefined class disease. In an example, “Viagra”, a drug used to treat erectile dysfunction. Consequently, such drug may be tagged “approved” for the biomedical entity: “erectile dysfunction”. However, “Viagra” may have a potential of use in treating pulmonary arterial hypertension, and thus, may be tagged with the tag “potential” for the biomedical entity: “Pulmonary Arterial Hypertension”. Consequently, tagging the biomedical entities belonging to the predefined class drug provides an efficient and informed way of identifying biomedical entities. Furthermore, it will be appreciated that the tag associated with a biomedical entity in the class: drug, may be determined in respect to another biomedical entity. In an example, a biomedical entity such as “Aspirin” may be tagged with the tag “approved” in respect to the biomedical entity: “Fever”. However, “Aspirin” may be tagged with a tag “potential” in respect to biomedical entity: “Gastrointestinal bleeding”.

The method further comprises identifying at least one pair of biomedical entities, from the plurality of extracted biomedical entities, having an association there between, wherein each biomedical entity of the at least one pair of biomedical entities belongs to different predefined classes. Specifically, the processing module is operable to identify at least one pair of biomedical entities, from the plurality of extracted biomedical entities, having an association there between, wherein each biomedical entity of the at least one pair of biomedical entities belongs to different predefined classes. Furthermore, characteristics of the extracted biomedical entities belonging to each of the predefined classes are analysed. Subsequently, biomedical entities, in the plurality of biomedical entities, with related functions and affects are paired in order to establish association there between. Additionally, biomedical entities in the at least one pair have a relation there between. Furthermore, the at least one identified pair of biomedical entities may have a direct or indirect relation with the biomedical entity of the user-input. In an instance when biomedical entity of the user-input may belong to the input class: target, a pair of biomedical entities belonging to the different predefined classes: disease and drug may be identified. In such an instance, the biomedical entity in the predefined class: disease may have a direct association with the biomedical entity of the user-input and the biomedical entity in the predefined class: drug might have an association with the biomedical entity of user-input through the biomedical entity in the predefined class: disease.

Optionally, the at least one pair of biomedical entities having the association there between comprises biomedical entities belonging to at least one of the following different predefined classes: target and drug, drug and disease, target and disease, pathway and disease, target and pathway. Specifically, in an instance when biomedical entities in the pair belong to the predefined classes: target and drug respectively, the biomedical entity belonging to the predefined class drug influences performance of the biomedical entity belonging to the predefined class target. Additionally, such an association might have a further association with another biomedical entity belonging to predefined class: disease. In a first example, a biomedical entity “EGFR” belonging to the predefined class: target may have an association with another biomedical entity “Trastuzumab” belonging to the predefined class: drug. Specifically, “Trastuzumab” influences EGFR levels in the cancer cells. In another instance, when biomedical entities in the pair belong to the predefined classes: drug and disease respectively, the biomedical entity belonging to the predefined class: drug may be used in treatment of the biomedical entity belonging to the predefined class: disease. Referring to the first example, the biomedical entity “Trastuzumab” belonging to the predefined class: drug may have an association with another biomedical entity “breast neoplasm” belonging to the predefined class: disease. The drug “Trastuzumab” may be used in treatment of the disease “breast neoplasm”. In yet another instance, when biomedical entities in the pair belong to the predefined classes: target and disease respectively, the biomedical entity belonging to the predefined class: disease may be caused because of abnormal functioning of the biomedical entity belonging to the predefined class: target. Furthermore, the biomedical entity belonging to the predefined class: disease may cause abnormal functioning of the biomedical entity belonging to the predefined class: target. Referring to the first example, the biomedical entity “EGFR” belonging to the predefined class: target may have an association with the biomedical entity “breast neoplasm” belonging to the predefined class: disease. Specifically, “breast neoplasm” associates with amplification (namely, overexpression) of EGFR genes. In an instance, when biomedical entities in the pair belong to the predefined classes: pathway and disease respectively, the biomedical entity belonging to the predefined class: disease may be caused due to an irregular event caused in the associated biomedical entity belonging to the predefined class: pathway. Referring to the first example, the biomedical entity “breast neoplasm” belonging to the predefined class: disease may be associated with a biomedical entity “mitogen-activated protein kinase (MAPK) signaling” belonging to the predefined class: pathway. Specifically, abnormal regulation of MAPK signaling pathway may be involved in the occurrence and progression of “breast neoplasm”. In yet another instance, when biomedical entities in the pair belong to the predefined classes: target and pathway respectively, the biomedical entity belonging to the predefined class: target may get affected because of an irregular or abnormal event in route of operation thereof. Such route of operation may be a biomedical entity belonging to the predefined class pathway. Furthermore, the association between the biomedical entity belonging to the predefined class: target and the biomedical entity belonging to the predefined class: pathway may be directly or indirect. Referring to the first example, the biomedical entity “EGFR” belonging to the predefined class: target may have an association with the biomedical entity “mitogen-activated protein kinase (MAPK) signalling” belonging to the predefined class: pathway. Specifically, overexpression of “EGFR” level in “MAPK signalling” pathway may cause occurrence and propagation of “breast neoplasm”.

Optionally, identifying at least one pair of biomedical entities, from the plurality of extracted biomedical entities, having an association there between comprises identifying potential associations between at least one pair of biomedical entities. Furthermore, potential associations refer to associations that are derived from existing associations. In an instance, a biomedical entity belonging to the predefined class: drug may be associated to a second biomedical entity belonging to the predefined class: disease. Additionally, a third biomedical entity belonging to the predefined class drug may be a potential drug for treating the second biomedical entity. Consequently, the second biomedical entity and the third biomedical entity may have a potential association there between. In an example, a first biomedical entity “Viagra”, belonging to a predefined class drug, may be used to treat a second biomedical entity “erectile dysfunction”, belonging to a predefined class disease, may have an association there between. Furthermore, the first biomedical entity “Viagra” may also be used in treating a third biomedical entity “pulmonary arterial hypertension”, belonging to the predefined class disease. Consequently, the first biomedical entity “Viagra” and the third biomedical entity “pulmonary arterial hypertension” may have a potential association there between. Additionally optionally, the biomedical entities in each of the predefined classes may be scored based on an importance score thereof.

Optionally, potential associations between biomedical entities belonging to the predefined classes: drug and target, is identified by processing of data records associated with such biomedical entity. Specifically, for a biomedical entity belonging to the predefined class: drug, plurality of potential biomedical entities belonging to the predefined class: target are extracted from the data records. Consequently, druggability of each of the plurality of potential biomedical entities is analysed and the plurality of potential biomedical entities are filtered. Subsequently, the filtered plurality of potential biomedical entities are analysed based on properties associated therewith. Based on the properties associated with each of the plurality of potential biomedical entities belonging to the predefined class: target, potential association of the biomedical entity belonging to the predefined class: target is extracted with the biomedical entity belonging to the predefined class: target.

Optionally, the properties associated with each of the filtered plurality of biomedical entity belonging the predefined class: target, include likelihood of mutations, number of available gene expression studies and gene ontologies, and number of pathways associated with the biomedical entity. Specifically, the likelihood of mutations comprises the likelihood of genetic and somatic mutations in the biomedical entity. Furthermore, optionally, such identification of potential associations between biomedical entities belonging to the predefined classes: drug and target may be identified in an instance when either a target or drug is the input class.

Optionally, the method further comprises determining the importance score for each of the plurality of biomedical entities based on a first set of predetermined parameters. Furthermore, the processing module is operable to determine an importance score for each of the plurality of biomedical entities based on the first set of predetermined parameters. Specifically, importance factor of a biomedical entity may provide additional information regarding relevance of the biomedical entity with the user-input. Additionally, the importance score may further indicate researches and publications available associated to the biomedical entity. Furthermore, the importance score may also indicate most recent the biomedical entity in a predefined class. Optionally, the importance factor can be determined by applying a function on the first set of predetermined parameters. In an example, the first set of predetermined parameters may include number of PMID (PubMed Identifier), mechanism of action (MOA), number of associations of the biomedical entity and so forth. Furthermore, such first set of predetermined parameters may be different for biomedical entities in different predefined classes. In an instance, when the biomedical entity belongs to the predefined class: target, the first set of predetermined parameters for determining the importance score includes: a first subset of predetermined parameters and a second subset of predetermined parameters. The first subset of predetermined parameters includes: availability of approved drug for the target, availability of known structure of the target, presence of transmembrane helix, presence of genetic association and availability of gene expression data. The second subset of predetermined parameters includes number of PMID, number of antibody, number of monoclonal antibody, number of associated biomedical entities belonging to the predefined class pathways, number of associated biomedical entities belonging to the predefined classes drug and disease. Furthermore, the availability of approved drug for the target indicates availability of a known and tested drug for use on the target. Additionally, the availability of known structure of the biomedical entity indicates availability of information associated with anatomy of the biomedical entity. Moreover, the presence of transmembrane helix indicates presence of a membrane-spanning domain with a hydrogen-bonded helical configuration in the biomedical entity belonging to the predefined class target. Furthermore, the presence of genetic association indicates availability of information associated with genes and diseases related thereto with regard to the biomedical entity belonging to the predefined class target. Moreover, the availability of gene expression data relates to information associated with a process by which information from a gene is used in the synthesis of a functional gene product (such as, proteins, non-protein coding genes such as transfer RNA, small nuclear RNA). Specifically, a score of “1” (in case of presence of a parameter) or “0” (in case of absence of a parameter) is determined for each of the parameters in the first subset of parameters. Subsequently, an intermediate score may be calculated based on score of each of the parameters in the first subset of predetermined parameters. Furthermore, in the second subset of predetermined parameters, the number of PMID associated with the biomedical entity belonging to the predefined class: target indicates publications, literature and researches published with PubMed that have an association with the biomedical entity. Additionally, the number of antibody indicates count of proteins produced substantially by plasma cells that is used by immune system to neutralize pathogens such as bacteria and viruses. Moreover, the number of monoclonal antibody indicates number of antibodies that are made by identical immune cells that are all clones of a unique parent cell present in the biomedical entities belonging to the predefined class target. Furthermore, the importance score for the biomedical entity belonging to the predefined class target may be calculated based on each of: the intermediate score of the first subset of predetermined parameters, the second set of predetermined parameters. The importance score of the biomedical entity belonging to the predefined class target indicates druggability (ability to be treated) of the biomedical entity.

Optionally, in another instance, when the biomedical entity belongs to the predefined class: disease, the first set of predetermined parameters for determining the importance score includes: number of PMID, number of associations of the biomedical entity with biomedical entities of the predefined classes except the input class, number of grants specifying funding from government organizations such as World Health Organization, Department of Biotechnology and the like. Additionally, the importance factor may also depend on number of active and completed clinical trials associated with treatment of the biomedical entity belongs to the predefined class disease. Consequently, the aforementioned first set of predetermined parameters for determining the importance score for the biomedical entity belonging to the predefined class disease indicates cure and risk factors involved in treatment associated with the biomedical entity. Furthermore, the importance factor for the biomedical entity belonging to the predefined class disease indicates common or rare occurring of the biomedical entity in one or more subjects.

Optionally, in yet another instance, when the biomedical entity belongs to the predefined class: pathway, the first set of predetermined parameters for determining the importance score includes: number of PMID and number of associations with other biomedical entities belonging to the predefined classes except the input class. Furthermore, the number of PMID of such biomedical entity indicate publications and research published with PubMed related to one or more disease and drugs associated with the biomedical entity belonging to the predefined class pathway. Additionally, the number of PMID of such biomedical entity may further indicate publications and research related to mechanism of the biomedical entity. Moreover, the importance factor of the biomedical entity belonging to the predefined class pathway may indicate probability of association thereof with one or more diseases and drugs.

Optionally, in an instance when the biomedical entity belongs to the predefined class: drug, the first set of predetermined parameters for determining the importance score includes: number of PMID, number of associations with other biomedical entities belonging to the predefined classes except the input class, mechanism of action and pharmacokinetics. Specifically, PMID of the biomedical entity belonging to the predefined class: drug may indicate number of PubMed publications and research related to the drug. Furthermore, number of associations of the biomedical entity with biomedical entities of predefined classes except the input class may indicate applicability of the biomedical entity. Furthermore, mechanism of action of the biomedical entity relates to process of working with associated biomedical entities. Moreover, pharmacokinetics may be used for determining the importance score by applying “Lipinski's rule of five elements”. Specifically, “Lipinski's rule of five elements” states that an orally active drug has no more than one violation in the following criteria: no more than five hydrogen bond donors, no more than ten hydrogen bond acceptors, a molecular mass less than 00 Daltons and an octanol-water partition coefficient log P not greater than five. Consequently, the aforementioned first set of predetermined parameters for determining the importance score for the biomedical entity belonging to the predefined class drug validate “drug likeness” (namely, ability to work as drug) of the biomedical entity belonging to the predefined class drug. Consequently, the importance score of a biomedical entity relates to aforementioned parameters associated to respective the biomedical entity. Specifically, the at least one pair of biomedical entities may have importance score related to each of the biomedical entities associated therein.

Optionally, the method further comprises determining a weightage score for each of the associations between the at least one pair of biomedical entities based on a second set of predefined parameters. Furthermore, the processing module is operable to determine the weightage score for each of the association between the at least one pair of biomedical entities based on the second set of predefined parameters. Specifically, a high weightage score indicates a well-known and approved association between the at least one pair of biomedical entities. Additionally, optionally, a low weightage score indicates a potential association. Furthermore, weightage score may affect representation scheme of the association between the at least one pair of biomedical entities based on a second set of predefined parameters. Moreover, the second set of predefined parameters may be different for each of the association between the at least one pair of biomedical entities. In an instance, when the biomedical entities in at least one pair belong to the predefined classes: drug and target, the second set of predefined parameters includes: hypergeometric association score based on PMID, assay count, animal model and well-known associations between the biomedical entities belonging to the predefined classes: drug and target. Furthermore, hypergeometric association score may be a method to identifying closest and known associations using hypergeometric distribution of the biomedical entities belonging to the predefined classes: drug and target based on number of PubMed publications thereof. Moreover, assay count refers to clinical trial count of the biomedical entity belonging to the predefined class: drug and testing thereof on the associated biomedical entity belonging to the predefined class drug. Furthermore, the animal model indicates that the biomedical entity belonging to the predefined class drug has been tested on the biomedical entity belonging to the predefined class target in an animal body. Moreover, the parameter well-known association between the biomedical entities belonging to the predefined classes: drug and target may act as a punishing score for the weightage score of the association thereof. Additionally, punishing score used herein refers to a negative score that reduces the related weightage score when association thereof may be well-known. Beneficially, by way of punishing score, less-known associations between the at least one pair of biomedical entities are emphasized.

Optionally, in another instance when the at least one pair of biomedical entities in the association may belong to the predefined classes: drug and disease respectively. Additionally, at such an instance the second set of predefined parameters includes: hypergeometric association score based on the targets, clinical trials and well-known associations between the biomedical entities belonging to the predefined classes: drug and disease. Furthermore, hypergeometric association score may be a method to identifying closest and known associations between biomedical entities belonging to the predefined class: drug and disease using hypergeometric distribution of the biomedical entities based on association thereof with biomedical entities belonging to the predefined class: target. Moreover, number of completed and ongoing clinical trials for biomedical entity belonging to the predefined class: drug related to the biomedical entity belonging to the predefined class: disease. Furthermore, well-known association between biomedical entities belonging to the predefined classes: drug and disease may act as punishing score for the weightage score of the association.

Optionally, in another instance, when the at least one pair of biomedical entities in the association may belong to the predefined classes: target and disease respectively. Additionally, at such an instance the second set of predefined parameters includes: hypergeometric association score based on PMID, and well-known associations between the biomedical entities belonging to the predefined classes: target and disease. Specifically, hypergeometric association score may be a method to identifying closest and known associations using hypergeometric distribution of the biomedical entities belonging to the predefined classes: target and disease based on number of PubMed publications thereof. Moreover, availability of well-known associations between the biomedical entities belonging to the predefined classes: target and disease may act as punishing score for the weightage score of the association thereof.

Optionally, in an instance, when the at least one pair of biomedical entities in the association may belong to the predefined classes: pathway and disease respectively. Additionally, at such an instance the second set of predefined parameters includes: hypergeometric association score based on the targets, well-known associations between the biomedical entities belonging to the predefined classes: pathway and disease. Furthermore, hypergeometric association score may be a method to identifying closest and known associations between biomedical entities belonging to the predefined class: pathway and disease, using hypergeometric distribution of the biomedical entities based on association thereof with biomedical entities belonging to the predefined class: target. Additionally, well-known associations between the biomedical entities belonging to the predefined classes: pathway and disease may act as punishing score for the weightage score of the association thereof.

Optionally, in another instance, when the at least one pair of biomedical entities in the association may belong to the predefined classes: target and pathway. Additionally, at such an instance the second set of predefined parameters includes: hypergeometric association score based on PMID, and well-known associations between the biomedical entities belonging to the predefined classes: target and pathway. Specifically, hypergeometric association score may be a method to identifying closest and known associations using hypergeometric distribution of the biomedical entities belonging to the predefined classes: target and pathway based on number of PubMed publications thereof. Moreover, availability of well-known associations between the biomedical entities belonging to the predefined classes: target and pathway may act as punishing score for the weightage score of the association thereof.

Consequently, the extracted plurality of biomedical entities related to the user-input and belonging to the predefined classes except the input class are paired based on association thereof. Subsequently, the paired biomedical entities belonging to the predefined classes except the input class are associated with the biomedical entity of the user-input.

As mentioned previously, the method further comprises: mapping the plurality of biomedical entities to the biomedical entity of the user-input. Specifically, the processing module is operable to map the plurality of biomedical entities to the biomedical entity of the user-input. Furthermore, the plurality of biomedical entities are paired and subsequently mapped to the biomedical entity user-input. Consequently, a mapped association of biomedical entities belonging to each of the predefined classes is determined. At an instance when the biomedical entity of the user-input belongs to the predefined class target, the biomedical entity of the user-input is mapped with the at least one pair of biomedical entity belonging to the predefined classes: disease, pathway and drug. At another instance when the biomedical entity of the user-input belongs to the predefined class disease, the biomedical entity of the user-input is mapped with the at least one pair of biomedical entity belonging to the predefined classes: target, pathway and drug. At yet another instance when the biomedical entity of the user-input belongs to the predefined class pathway, the biomedical entity of the user-input is mapped with the at least one pair of biomedical entity belonging to the predefined classes: target, disease and drug. At another instance when the biomedical entity of the user-input belongs to the predefined class drug, the biomedical entity of the user-input is mapped with the at least one pair of biomedical entity belonging to the predefined classes: target, disease and pathway. Furthermore, the mapped association of biomedical entities belonging to each of the predefined classes may be represented in form of a network, a tabular structure and so forth. Beneficially, the mapped association of biomedical entities belonging to each of the predefined classes may provide information related to biomedical entities belonging to any of the predefined classes, wherein the biomedical entities have an association there between.

Furthermore, there is disclosed a computer readable medium containing program instructions for execution on a computer system, which when executed by a computer, cause the computer to perform method steps for mapping biomedical entities, wherein each of the biomedical entities belongs to one of a predefined class: target, disease, pathway, and drug. The method comprises the steps of providing a user-input of a biomedical entity belonging to one of the predefined classes, wherein the predefined class of the biomedical entity defines an input class; extracting a plurality of biomedical entities related to the biomedical entity of the user-input from existing data sources, wherein the plurality of biomedical entities belong to predefined classes except the input class; identifying at least one pair of biomedical entities, from the plurality of extracted biomedical entities, having an association there between, wherein each biomedical entity of the at least one pair of biomedical entities belongs to different predefined classes; and mapping the plurality of biomedical entities to the biomedical entity of the user-input. Optionally, the computer readable medium comprises one of a floppy disk, a hard disk, a high capacity read only memory in the form of an optically read compact disk or CD-ROM, a DVD, a tape, a read only memory (ROM), and a random access memory (RAM).

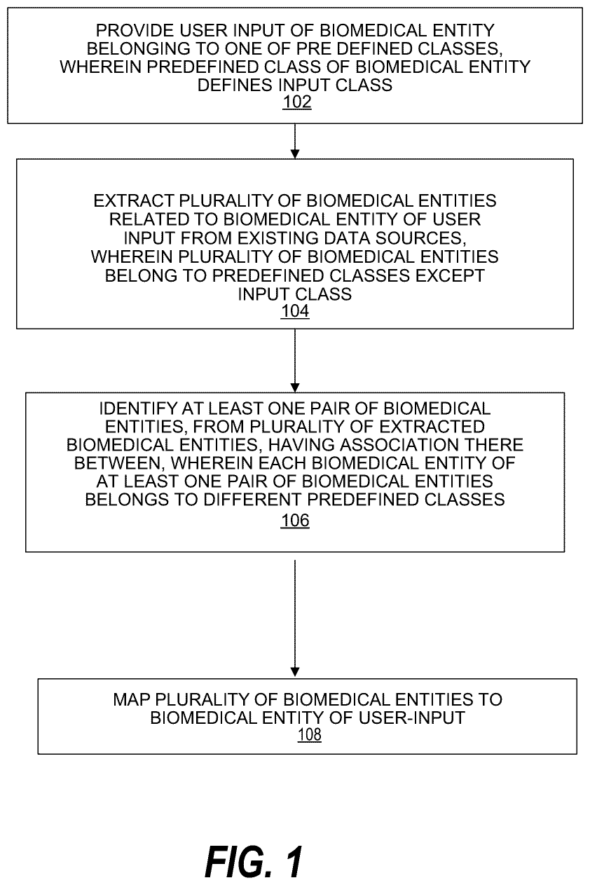

Referring to FIG. 1, illustrated are steps of a method 100 of mapping biomedical entities, in accordance with an embodiment of the present disclosure. Moreover, each of the biomedical entities belongs to one of a predefined class: target, drug, disease, and pathway. At a step 102, a user input of a biomedical entity belonging to one of the predefined classes is provided. Additionally, the predefined class of the biomedical entity defines an input class. At a step 104, plurality of biomedical entities related to the biomedical entity of the user input are extracted from existing data sources. Furthermore, the plurality of biomedical entities belongs to predefined classes except the input class. At a step 106, at least one pair of biomedical entities, having an association there between, is identified from the plurality of extracted biomedical entities. Moreover, each biomedical entity of the at least one pair of biomedical entities belongs to different predefined classes. At a step 108, the plurality of biomedical entities are mapped to the biomedical entity of the user-input.

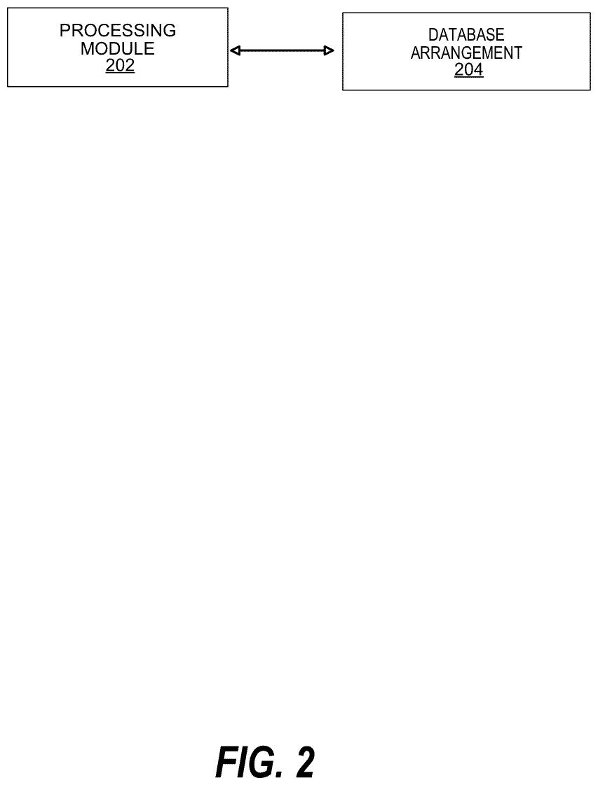

Referring to FIG. 2, illustrated is a block diagram of a system 200 that maps biomedical entities, in accordance with an embodiment of the present disclosure. The system 200 comprises a processing module 202 operable to map the biomedical entities. The processing module 200 is further operable to receive a user-input of a biomedical entity belonging to a predefined class. Subsequently, the processing module is operable to map plurality of biomedical entities to the biomedical entity of the user-input, wherein the plurality of biomedical entities have an association with the biomedical entity of the user-input. The system 200 further comprises a database arrangement 204 communicably coupled to the processing module 202. The database arrangement 204 is operable to store existing data sources.

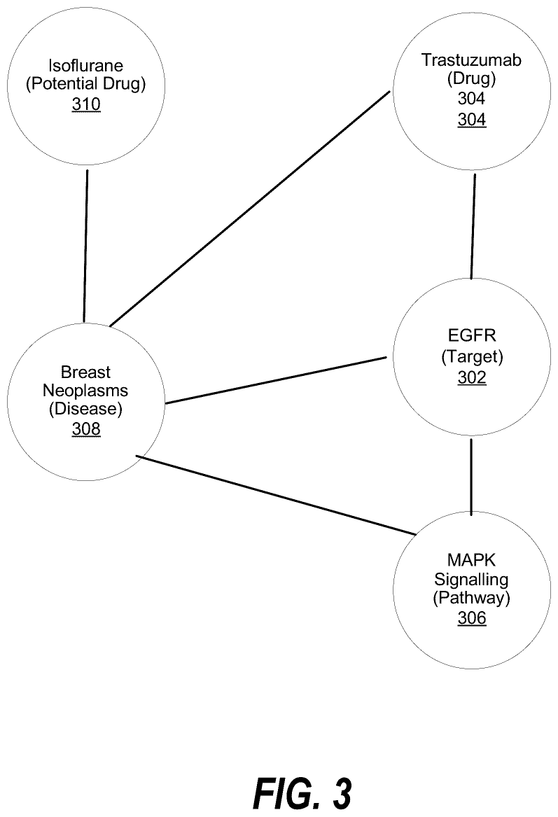

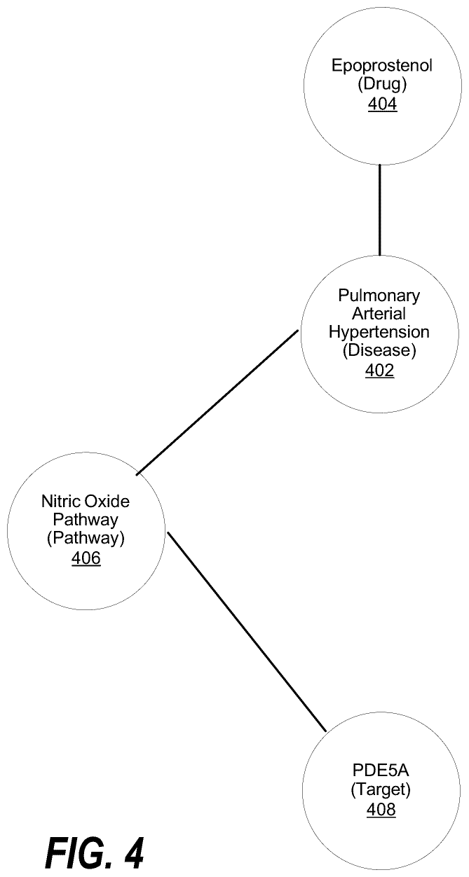

Referring to FIGS. 3 and 4, there are shown schematic illustrations of mapped associations of biomedical entities, in accordance with exemplary implementations of the present disclosure. The exemplary implementations relate to the method 100 (of FIG. 1).

In FIG. 3, a user input 302 of a biomedical entity “EGFR” belonging to the predefined class: target is provided. Consequently, a plurality of biomedical entities related to the biomedical entity of the user-input 302, such as biomedical entities “Trastuzumab” 304 belonging to the predefined class: drug, “MAPK signaling” 306 belonging to the predefined class: pathway, and “Breast Neoplasm” 308 belonging to the predefined class: disease, are extracted existing data sources. Subsequently, at least one pair of biomedical entities, such as the pair of biomedical entities 304 and 308, and 306 and 308, having an association there between is identified. Subsequently, the biomedical entities 304, 306, 308 are mapped to the biomedical entity of the user-input 302.

Optionally, the biomedical entity of user-input 302 acts as central element in mapped representation of biomedical entities. Optionally, a potential association may be identified between the biomedical entity “Breast Neoplasm” 308 belonging to the predefined class: disease and biomedical entity: Isoflurane 310 belonging to the predefined class: drug. Specifically, Isoflurane 310 is a potential drug associated to the biomedical entity Breast Neoplasm 308.

In FIG. 4, a user-input 402 of biomedical entity “Pulmonary Arterial Hypertension” belonging to the predefined class: disease, is provided. Therefore, in such a case, the input class is defined as the disease. Consequently, a plurality of biomedical entities related to the biomedical entity of the user-input 302, such as: Epoprostenol 404 belonging to the predefined class: drug and Nitric Oxide Pathway 406 belonging to the predefined class: pathway, are extracted. Furthermore, the biomedical entity Nitric Oxide Pathway 406 is associated to biomedical entity PDE5A 408 belonging to the predefined class: target.

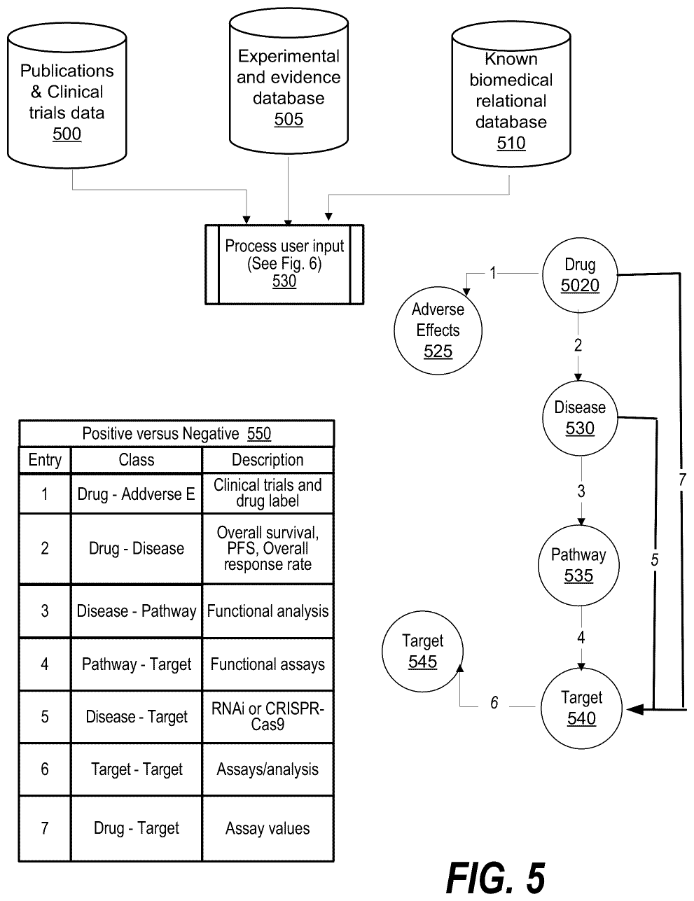

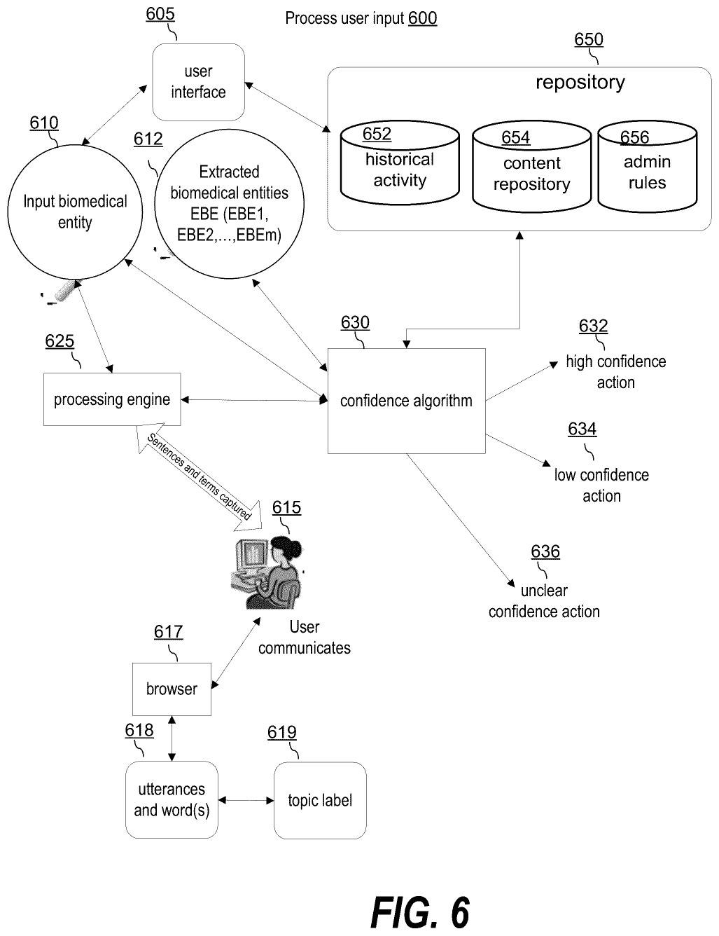

FIG. 5 depicts aspects of an embodiment of the invention described herein. When a user utilizes the user interface provided, the process performs the process user input routine (see FIG. 6 and corresponding text for processing details). When the user specifies a medical entity of interest, information related to the medical entity itself and other information related to other medical entities belonging predefined classes different from the class of the medical entity itself is retrieved from publications and clinical trials data store 500, from experimental and evidence database data store 505, and from known biomedical relational database data store 510. In an example embodiment, when the user has specified a drug 520, a target-target association and a drug-adverse event association are included as biomedical entities. All documents associated with specific biomedical terms are extracted from the referenced data stores, 500, 505, and 510, which are then classified as positive or negative, or supportive or unsupportive. These associations are further enhanced through the extraction of experimental values using the positive versus negative 550 table extraction model. This automated process enriches the biomedical associations, providing valuable insights. The positive and negative associations, along with their corresponding experimental values, can be leveraged to prioritize and filter desired biomedical associations. In an embodiment, to ensure easy accessibility, these associations and experimental values may be stored in a No Structured Query Language (SQL) database, allowing for quick retrieval whenever needed. Associations may be dynamically extracted from the NoSQL database and entered in a knowledge graph (KG) as dynamically as needed according to user requests. In an example scenario, when the user has identified a drug 520 for analysis, entry 1 may use the class drug—adverse effects 525, which may have been extracted from clinical trials according to drug labels. Entry 2 may use the class drug—disease 530, which may use terms such as, overall survival, Progression-free survival (PFS), overall response rate, disease-free survival (DFS). Entry 3 may use class disease—pathway 535, which may use terms such as, functional assays, pathway inhibition studies, pharmacological intervention, pathway activation analysis. Entry 4 may use class pathway—target 540, which may use terms such as, functional assays. Entry 5 may use class disease—target 545, which may use terms such as, RNAi, CRISPER-Cas9. Entry 6 may use class target—target 540, which may use terms such as, co-immunoprecipitation, yeast, two-hybrid assay, proximity ligation assay (PLA), bioluminescence resonance energy transfer (BRET) assay, surface plasmon resonance (SPR) analysis. Entry 7 may use class drug—target 545, which may use terms such as, IC₅₀³, Ki, Kd, drug target inhibition assay values.

An aspect of the invention is described in an example scenario. The user 615 is an employee of a drug company looking for other usages of a specific pharmacological product, a drug. In an embodiment, the user 615 uses one or more sentences to communicate the request. Each sentence is referred to as an utterance. Each utterance includes keywords and phrases. The keywords and phrases may be sufficient for the system to understand and proceed with the request. The process of determining the reason for the communication is called utterance labeling. Once an utterance is labeled, the processing of the request by the processing engine 625 occurs. If the labeling is correct, then the processing is likely to follow a path that meets the needs of the user 615. If the labeling is not correct, then there is a significant chance that the user will receive not useful results.

In order to label the utterances correctly, a history of user access is collected. In some embodiments, the history, the sentences, and the keywords are captured in a repository 650. The disposition could be made during the communication. In another embodiment, it could be after the user verifies the information received satisfies the request via a user survey. While the user 615 communicates with processing engine 625 various information is collected. The information may be as simple as topic words the user 615 uses with the processing engine 625. The processing engine 625 receives the collected information according to an input biomedical entity 610 that the user 615 requested for a focus of analysis. The processing engine 625 utilizes confidence algorithm 630 which interfaces with repository 650. The repository 650 may have various elements. The elements may include, but are not limited to, for example, historical activity 652 that captured other search terms used in the past for similar content stored in content repository 654, and admin rules 656 that are followed when interfacing with repository 650. The confidence algorithm 630 associates at least one input biomedical entity 610 from the utterances with the topic label 619 and characterizes the at least one input biomedical entity 610 with the topic label 619. In addition, the confidence algorithm 630 extracts biomedical entities EBE (EBE1, EBE2, . . . , EBEm) 612 belonging to predefined classes different from the predefined class of the input biomedical entity. The confidence algorithm 630 attempts to evaluate if the topic label 619 should be revised to reflect a predicted improvement in labeling and biomedical entity determination. Consideration may include historical activity 652 that includes terms users have used in the past while requiring the specific request or dialog act, such as, for example, add <term> as an entry under class disease. The similar content and a characterization of the similar content may be in content repository 654 that may include other features being searched. The confidence algorithm 630 utilizes the at least one input biomedical entity 610 and topic label 619 characterization to predict a confidence level of adjusting of the topic label 619 based on changing the at least one input biomedical entity 610 associated with the topic label 619. The change may be an addition of one input biomedical entity, a change of a second input biomedical entity, an addition of a third input biomedical entity, a deletion of a fourth input biomedical entity, and etc.

The confidence algorithm 630 may apply various admin rules 656 based on different optimization rules. The rules could be by target class, processing related to a specific repository, a mapping from one class to another class, and the like. Using the admin rules 656, the confidence algorithm 630 may utilize some type of statistical assessment to predict if a change to the topic label 619 should be made. When the confidence algorithm 630 determines that a missing keyword has a high probability of improving topic label 619, the confidence algorithm performs a high confidence action 632, such as, for example, but not limited to, adding additional information to content repository 654 under a predicted improvement to labeling category, updating or changing content in topic label 619, making a recommendation to change the topic label 619, and the like. Those keywords that improve topic label 619 are called input biomedical entities and are referenced as at least one input biomedical entity 610. However, if the confidence algorithm 630 determines that adding a missing keyword has a low probability of improving topic label 619, the confidence algorithm 630 performs a low confidence action 634, such as, for example, but not limited to, making a determination of not adding a missing input biomedical entity to the at least one input biomedical entity keyword 610 associated with the topic label 619. Alternatively, the missing keyword may be added to the content repository 654 under a not predicted to improve labeling category, a revise class categorization, and the like. If the confidence algorithm 630 determines that a missing keyword has an unclear probability of improving topic label 619, the confidence algorithm 630 performs an unclear confidence action 636, such as, for example, but not limited to, recording related information in historical activity 652. The confidence algorithm 630 may have an Artificial Intelligence (AI) component that learns which terms are relevant and utilizes a feedback loop adding new evaluations and new results to determine which terms are relevant. The feedback loop would have expected advantages, such as, speeding up processing time, improving user satisfaction and increasing the quality of the keywords in the topic label 619 to improve its accuracy. Having an existing keyword mapping to a label and a synonym of the existing keyword used in a similar utterance would be an example where a high confidence action 632 would be taken.

The historical activity 652 may be retrieved as well as the information from the content repository 654 to find associations between the usages. Natural language processing (NLP) may be applied to the historical activity 652, to the at least one input biomedical entity 610, and the content repository 654 to categorize each of the at least one input biomedical entity 610 and associate them with the topic label 619 and identify other biomedical entities having a predetermined class different from the at least one input biomedical entity class 612. Deep analytic analysis and artificial intelligence technologies may be used to adjust the categorization. Feedback from Subject Matter Experts (SMEs), and other user feedback may be used to tune the characterization and form a confidence level or ranking that the at least one input biomedical entity 610 affect the labeling of the topic label 619. In most cases, adding any keyword from the at least one input biomedical entity 610 to the topic label 619 is unlikely to change the topic label 619. However, adding some terms may have unwanted side effects. For example, some keywords may not relate to the specific biomedical entity or cause an undesirable association. Some embodiments may have different processes related to the at least one input biomedical entity 610 based on different criteria. The actions that follow depend on the confidence level and the admin rules 656.

The label is a classification that may indicate a drug, a disease, a target, or a pathway. The identified topic label 619 for the utterances and word(s) 618 communicated by the user 615 may be provided to the processing engine 625 via any of the communication technologies. It could be, for example, a description of steps to follow to land on the topic label 619 by utilizing the browser 617. The repository 650 may be a database management system (DBMS) supporting indexing, queries, and other typical database features. It could be any data store for recording and retrieving data. The repository 650 may include various elements, for example, but not limited to, historical activity 652 that records a history of interactions by different users by various methods, a content repository 654, that identifies, for example, biomedical entities and associates the biomedical entities with web pages, user browser activity when reaching web pages, and admin rules 656 that may determine policies for capturing information, rules for changing web pages, and the like. The repository 650 may have default rules for tracking of input biomedical entities and associating input biomedical entities with web pages. The repository 650 may be adaptive and may automatically adjust based on feedback via artificial intelligence (AI) technology. Although the user interface depicted in FIG. 6 is browser 617, any user interface may be used. The user interface may provide a GUI where the user inputs parameters as menu entries, command line entries, scripts entries, configuration files, .xml files, or any other means of providing the required information. In some embodiments, the system may derive the required information from a history of accesses kept by the browser 617. The browser 617 or the search engine used by the customer 616 may provide Application Programming Interfaces (APIs) such as a representational state transfer (REST) to support retrieving the browser 617 search history. As the system may keep track of information extracted from the search history to identify at least one input biomedical entity 610 and determine an initial confidence associated with the identified at least one input biomedical entity 610 based on patterns of access and queries. The processing engine 625 uses confidence algorithm 630 to access the repository 650 and to characterize the at least one input biomedical entity 610. The characterization identifies at least one input biomedical entity 610 to form an at least one topic label 619 characterization. The user interface may include various options, such as, for example, use only data from the repository, refresh the repository information changed from the external sources [500, 505, 510], and replace content in the repository based on current processing engine 625. Utilizing the option carefully allows a user to gather information from selected external sources and select specific biomedical entities for closer interactive analysis with dynamically constructed KB based around a dynamically selected biomedical entity.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

The present invention may be a system, a method, and/or a computer program product at any possible technical detail level of integration. The computer program product may include a computer readable storage medium (or media) having computer readable program instructions thereon for causing a processor to carry out aspects of the present invention.

A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an,” the same holds true for the use in the claims of definite articles.

What is claimed is:

1. A method for dynamically identifying associations between a first biomedical entity and a second biomedical entity comprising:
receiving the first biomedical entity via a user-interface;
mapping the first biomedical entity to a first class in a set of predefined classes wherein each entry in the set of predefined classes is one of a target, a disease, a pathway, and a drug;
extracting a plurality of biomedical entities related to the first biomedical entity from existing data sources, wherein the plurality of biomedical entities belong to one of the predefined classes except the first class;
storing the extracted plurality of biomedical entities in a repository;
identifying at least one pair of biomedical entities, from the plurality of extracted biomedical entities, and wherein the at least one pair has a first entry and a second entry with the first entry having an association with the second entry, and wherein the second entry belongs to one of the predefined classes except the first class; and
presenting, by the user-interface, a representation of the at least one pair of biomedical entities.

2. The method of claim 1, further comprising:
responsive to a user utilizing the user-interface and selecting the second entry for focused analysis, constructing a knowledge graph from the repository with a focus on biomedical entities related to the second entry; and
presenting, by the user-interface, a representation of the knowledge graph.

3. The method of claim 2, wherein the representation further comprises:
providing positive versus negative information in the knowledge graph associated with branches of the knowledge graph.

4. The method of claim 1, further comprising:
identifying at least one potential association between a selected pair of potential biomedical entities of the identified at least one pair of extracted biomedical entities;
analyzing drugability of at least one potential biomedical entity in the selected pair of potential biomedical entities; filtering the at least one potential biomedical entity;
analyzing the filtered at least one potential biomedical entity based on properties associated therewith;
extracting the at least one potential association between the selected pair of potential biomedical entities from the repository; and
dynamically presenting, by the user-interface, the at least one potential association between the selected pair of potential biomedical entities.

5. A computer system for dynamically identifying associations between a selected biomedical entity belonging to a first class and a different biomedical entity belonging to a different class from the first class that maps biomedical entities, wherein each of the biomedical entities belongs to one of a plurality of predefined classes: target, disease, pathway, and drug, wherein the system comprises:
a database arrangement configured to store existing data sources; and
a processing module communicably coupled to the database arrangement, the processing module operable to:
receive a user-input, using a user-interface, wherein the user-input is a biomedical entity belonging to one of the predefined classes, wherein the predefined class associated with the biomedical entity of the user-input defines an input class, wherein the input class is one of: target, disease, pathway, and drug;
extract a plurality of biomedical entities related to the biomedical entity of the user-input from the existing data sources, wherein the plurality of biomedical entities belong to one of the predefined classes except the predefined class identical to the input class;
identify at least one pair of biomedical entities, from the plurality of extracted biomedical entities, having an association there between, wherein each biomedical entity of the at least one pair of biomedical entities belongs to different predefined classes except the predefined class identical to the input class, wherein characteristics of the plurality of extracted biomedical entities belonging to each of the different predefined classes are analysed such that the biomedical entities with related functions and affects are paired in order to establish association there between, wherein the biomedical entities in the at least one pair have a relation there between, and wherein the at least one identified pair of biomedical entities belonging to different predefined classes except the predefined class identical to the input class have a direct or indirect relation with the biomedical entity of the user-input belonging to the input class; wherein identifying at least one pair of biomedical entities, from the plurality of extracted biomedical entities, having an association there between comprises:
identify at least one potential association between two biomedical entities of the plurality of extracted biomedical entities, wherein the identification of the at least one potential association between the two potential biomedical entities comprises:
analyze drugability of the at least one potential biomedical entity;
filter the at least one potential biomedical entity;
analyze the filtered at least one potential biomedical entity based on properties associated therewith;
extract the at least one potential association between the two potential biomedical entities to
obtain the at least one pair of biomedical entities;
map the at least one identified pair of biomedical entities belonging to different predefined classes except the predefined class identical to the input class to the biomedical entity of the user-input belonging to the input class; and
represent, on the user-interface, the at least one identified pair of biomedical entities belonging to different predefined classes except the predefined class identical to the input class that are mapped to the biomedical entity of the user-input belonging to the input class, wherein the biomedical entity of the user-input is a central element in the map.

6. The system of claim 5, wherein the at least one pair of biomedical entities having the association there between comprises biomedical entities belonging to at least one of the following different predefined class pairs: target and drug, drug and disease, target and disease, pathway and disease, target and pathway.

7. The system of claim 5, wherein the processing module is operable to tag the biomedical entities in the drug predefined class with one of the tags: approved, investigational, combinational, and potential.

8. A method for dynamically identifying associations between a selected biomedical entity belonging to a first class and a different biomedical entity belonging to a different class from the first class by mapping biomedical entities, wherein each of the biomedical entities belong to one of a plurality of predefined classes: target, disease, pathway, and drug, wherein the method is implemented via a computer system comprising a database arrangement configured to store existing data sources and a processing module coupled to the database arrangement, wherein the method comprises:
receiving a user-input, using a user-interface, wherein the user-input is a biomedical entity belonging to one of the predefined classes, wherein the predefined class associated with the biomedical entity of the user-input defines an input class, wherein the input class is one of: target, disease, pathway, and drug;
extracting a plurality of biomedical entities related to the biomedical entity of the user-input from the existing data sources, wherein the plurality of biomedical entities belongs to one of the predefined classes except the predefined class identical to the input class;
identifying at least one pair of biomedical entities, from the plurality of extracted biomedical entities, having an association there between, wherein each biomedical entity of the at least one pair of biomedical entities belongs to different predefined classes except the predefined class identical to the input class, wherein characteristics of the plurality of extracted biomedical entities belonging to each of the different predefined classes are analysed such that the biomedical entities with related functions and affects are paired in order to establish association there between, wherein the biomedical entities in the at least one pair have a relation there between, and wherein at least one identified pair of biomedical entities belonging to different predefined classes except the predefined class identical to the input class have a direct or indirect relation with the biomedical entity of the user-input belonging to the input class; wherein identifying at least one pair of biomedical entities,
from the plurality of extracted biomedical entities, having an association there between comprises:
identifying at least one potential association between two biomedical entities of the plurality of extracted biomedical entities, wherein the identification of the at least one potential association between the two potential biomedical entities comprises:
analyzing drugability of the at least one potential biomedical entity;
filtering the at least one potential biomedical entity;
analyzing the filtered at least one potential biomedical entity based on properties associated therewith;
extracting the at least one potential association between the two potential biomedical entities; and
obtaining the at least one pair of biomedical entities;
mapping the at least one identified pair of biomedical entities belonging to different predefined classes except the predefined class identical to the input class to the biomedical entity of the user-input belonging to the input class; and
representing, on the user-interface, the at least one identified pair of biomedical entities belonging to different predefined classes except the predefined class identical to the input class that are mapped to the biomedical entity of the user-input belonging to the input class, wherein the biomedical entity of the user-input is a central element in the map.

9. The method of claim 8, wherein the at least one pair of biomedical entities having the association there between comprises biomedical entities belonging to at least one of the following different predefined class pairs: target and drug, drug and disease, target and disease, pathway and disease, target and pathway.

10. The method of claim 8, wherein the method further comprises tagging the biomedical entities in the drug predefined class with one of the tags: approved, investigational, combinational, and potential.
